(19) United States
(12) Plant Patent Application Publication
(10) Pub. No.: US 2004/0003443 P1
(43) Pub. Date: Jan. 1, 2004

(54) Hydrangea plant named 'White Robe'

(76) Inventor: Harrison Higaki, San Mateo, CA (US)

Correspondence Address: JAMES R. CYPHER, 405 14TH STREET, SUITE 1607, OAKLAND, CA, 94612

(21) Appl. No.: 10/187,135

(22) Filed: Jun. 28, 2002

(51) Int. Cl. A01H 5/00

(52) U.S. Cl. PLT/250

(57) ABSTRACT

A new and distinct cultivar of Hydrangea macrophylla (Thunb.) named ‘WHITE ROBE’ originated as a cutting taken from the cultivar ‘BUFFIE’. The cultivar ‘WHITE ROBE’ has white sepals at maturity and is distinguished from other similar cultivars of which the inventor is aware by the unique manner in which the inflorescence ages. As the mature sepals age and turn green they are highly resistant to burning and turning brown The new variety ‘WHITE ROBE’ further possesses the favorable characteristics of a compact growth habit and long lasting large inflorescences.

### BACKGROUND OF THE INVENTION

This invention relates to a new and distinct cultivar of the Saxifragaceae family. The botanical name of the plant is Hydrangea macrophylla (Thunb.). The varietal denomination is ‘WHITE ROBE’.

The new cultivar originated as a cutting taken from the variety ‘BUFFIE’. The cutting was taken on May 27, 1999. The variety ‘BUFFIE’ has pigmented sepals, and is typically grown under soil conditions with pH conditions that produce pink pigmentation. The new variety was first noticed, because it had white sepals under soil conditions that would have produced pink sepals in ‘BUFFIE’.

‘WHITE ROBE’ is distinguished from other varieties of Hydrangea that have white sepals of which the inventor is aware by the manner it which its sepals age.

The new cultivar ‘WHITE ROBE’ has been successfully asexually reproduced under controlled environmental conditions at a nursery in Half Moon Bay, Calif. under the direction of the inventor over a three year period with its distinguishing characteristics remaining stable.

‘WHITE ROBE’ is distinguished from other white varieties of Hydrangea of which the inventor is aware by the manner it which its sepals age. Sepal color of ‘WHITE ROBE’ is predominately white at maturity. Sepals start green, and turn white as they mature. Then as the florets begin to age, the sepals turn green again. The characteristic that distinguishes ‘WHITE ROBE’ from all other white varieties known to the inventor is that as the mature sepals age and turn green they are highly resistant to burning and turning brown, unlike many other Hydrangeas. This makes the flowers of the plant more attractive for longer periods of time.

The new variety of Hydrangea as described herein is further characterized by its compact growth habit, and its large, long lasting inflorescences.

Asexual reproduction was first accomplished when vegetative cuttings were taken from the initially selected plant. Examination of asexually reproduced, successive generations grown in at Half Moon Bay, Calif. show that the combination of characteristics as herein disclosed for ‘WHITE ROBE’ remains firmly fixed.

Asexual reproduction of successive generations at Half Moon Bay was achieved by taking vegetative cuttings from selected plants five times over three years. Each new generation retained the combination of characteristics as herein disclosed for ‘WHITE ROBE’.

### DESCRIPTION OF THE DRAWINGS

The accompanying drawings consist of color photographs that show the typical plant form, including the inflorescence, foliage, and sepals.

The plant shown is approximately 1 years old. The plant started out as a cutting, taken from the stem of a grown plant. Two leaves were attached to the stem cutting. The cutting was placed in a 4″ pot with hydrangea soil that would produce blue pigmentation in Hydrangeas that have pigmentation in their sepals. The cutting was placed under mist for 3 to 4 weeks until the cutting rooted. The cutting was then moved out of the misting area, but left under glass. Once the cutting produced roots, the plant was fertilized approximately twice a week. The plant was kept inside under glass for 4 to 5 more weeks and then moved outside, and fertilized once a week.

FIG. 1 is a view of the entire plant showing its form, compact growth habit, dark green foliage, inflorescence, and white sepals

FIG. 2 is a close-up view of the individual florets illustrating the unique white sepals which, as they age, generally turn green without burning.

FIG. 3 is a close-up view taken from above a relatively young inflorescence of the new cultivar showing its large inflorescence.

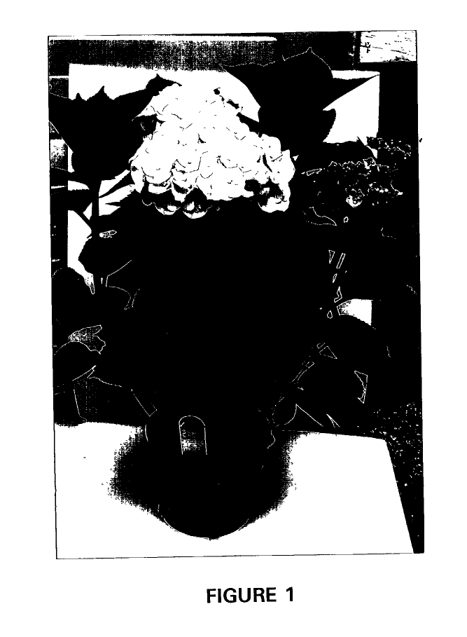

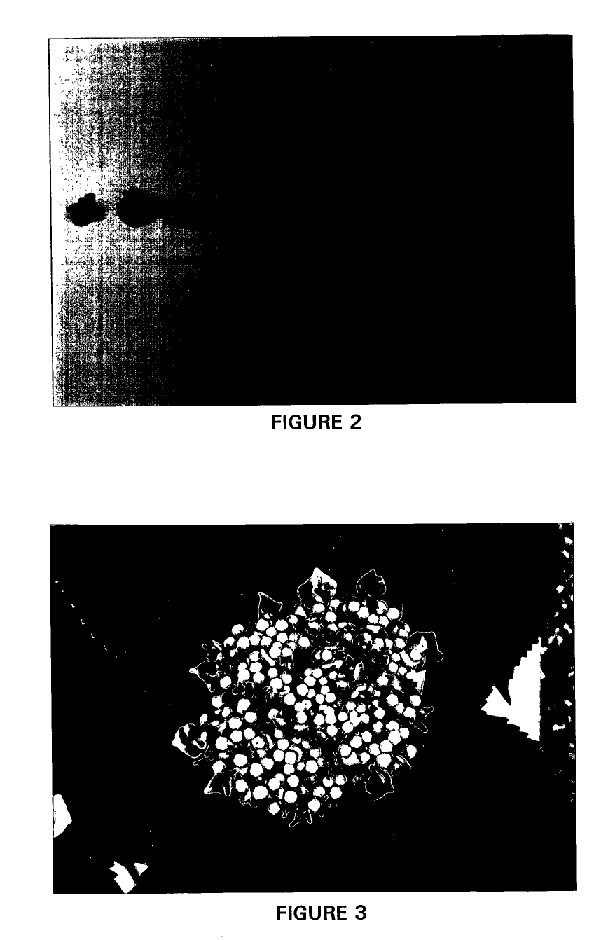

### DESCRIPTION OF THE NEW PLANT

‘WHITE ROBE’ has not been observed under all possible environmental conditions. The phenotype may vary significantly with variations in environment such as temperature, light intensity and day length. The following is a detailed description of a plant that started as a stem cutting and was grown in a 4″ pot under the prevailing daylengths at Half Moon Bay, Calif. The color determinations were made with The Royal Horticultural Society (R.H.S.) Colour Chart.

### THE PLANT

Origin: cutting.

Parent: Hydrangea macrophylla (Thunb.) ‘BUFFIE’.

Classification:

Botanic.—Hydrangea macrophylla (Thunb.) ‘WHITE ROBE’.

Commercial.—Florist Hydrangea ‘WHITE ROBE’.

Form: Upright, compact shrub.

Growth: Upright, vigorous growth habit. Inflorescence is large.

Stems: Lenticels are present; stems become woody as they age.

Foliage: Abundant.

Shape of leaf.—Elliptic with acute base and apex; margins are serrate.

Texture.—Glabrous; veins dominate on the underside of the leaf and are sunken on the leaf surface.

Color.—Mature leaves have an upper side that is R.H.S. 137 A (green group); under side is R.H.S. 147 D (yellow-green group).

### BUDS

Form: Globose; with 4 to 5 connate petals. Buds in the very center of the inflorescence are non-sepalous. The majority of buds have sepals.

Aspect: Smooth.

Arrangement: Borne on branched panicles.

Color: Buds and sepals are yellow-green, and whiten as the bud matures.

### INFLORESCENCE

Form: Paniculate. 100 or more individual flowers (florets) per inflorescence. Both sepalous florets and non-sepalous florets borne on same panicle with the sepalous florets hiding the non-sepaolous florets, when the inflorescence reaches maturity.

Size of inflorescence: Large. Individual inflorescence size is dependent on the number of florets.

Shape: Clusters of numerous small florets; sepalous florets are flat and overlapping one another. Sepals are persistent. Sepals elongate and greens as the inflorescence ages past maturity. Non-sepalous florets are inconspicuous and hidden by sepalous florets.

Appearance: Showy.

### FLORETS

General: Sepalous florets are perfect and complete, although petals, stamens and ovary generally fall off as floret approaches maturity.

Corrola: Generally there are 5 petals with 1 petal being wider than the other 4. In the sepalous florets the petals generally fall off before the inflorescence reaches maturity.

Reproductive organs:

Stamens.—8 to 10 stamens. Pollen is white. Filament is purple-blue and approximately 2 mm long. Anther is 1 mm long and is purple-blue when young and begins to green as floret approaches maturity.

Stigma.—Two to three-pronged stigma. Stigma is purple-blue.

Ovary.—Green in color.

Sepalous florets:

Number of sepals.—4 or 5 sepals per floret, usually 4.

Aspect of sepals.—Smooth.

Shape of sepals.—Reniform with acuminate apex. Edges often wavy when the floret is young.

Size of sepals.—Usually 1 dominant sepal, 2 smaller but equally-sized sepals, and 1 small sepal.

Coloration of sepals.—Predominately white at maturity. As inflorescence ages past maturity sepals green, generally without burning or turning brown.

Fruit: Brown and dry.

Seeds: Brown.

I claim:

1. A new and distinct hybrid plant variety of the Saxifragaceae family substantially as herein shown and described.
